(19) United States
(12) Patent Application Publication
(10) Pub. No.: US 2007/0286907 A1
(43) Pub. Date: Dec. 13, 2007

(54) Germicide composition

(76) Inventors: Gregg Siegel, San Antonio, TX (US); Phyllis Siegel, San Antonio, TX (US)

Correspondence Address: JOHN LEZDEY & ASSOCIATES, Suite 118, 2401 West Bay Drive, Largo, FL, 33770, US

(21) Appl. No.: 11/321,224

(22) Filed: Dec. 29, 2005

(51) Int. Cl. A61K 33/36

(52) U.S. Cl. 424/669

(57) ABSTRACT

A method and composition for removing biofilm, disinfecting and inactivating endotoxin which generates a safe level of molecular iodine that is free of persulfate ions. The composition can be used in households, industry, on medical equipment and skin surfaces.

### FIELD OF THE INVENTION

The present invention relates to a germicidal composition which also inactivates endotoxins. More particularly, there is provided a composition which generates a safe level of microbicidal molecular iodine and an oxidizing agent which is free of persulfate ions. There is also provided a method for inactivating endotoxin in solutions, medical devices and biological environments.

### BACKGROUND OF THE INVENTION

There are all types of germicidal agents which are used to disinfect medical devices and to remove biofilm. However, while the disinfectants are bactericidal, they have no effect on endotoxins. Endotoxins induce acute symptoms such as increase temperature and induce aches and malaise, initiate blood coagulation and influence the complement system and can exacerbate clinical signs and symptoms in a sick patient. The problem of endotoxin in medical apparatuses is acute, such as dialysis waterlines and dialysis machines where biofilms form.

Glutaraldehyde solution is commonly used for disinfecting medical devices and apparatus. However, glutaraldehyde has an obnoxious odor and is considered to be carcinogenic. Bleach is also used for this purpose and it is corrosive.

In an effort to protect the public from unsanitary surfaces in public restrooms, telephones and other surfaces which are contacted by the public, a number of methods have been developed. Moreover, the public has become further aware of transmitting various pathogens into the home. It has become desirable to provide a germicide which is bactericidal, pseudomonacidal, virucidal, tuberculacidal, fungicidal and can eliminate biofilms.

The presence of endotoxin, a component of the cell wall of gram-negative bacteria is perhaps a greater threat in causing blood stream infections than bacteria. Endotoxin standards in Europe is 0.5 EU/ml of endotoxin.

Bacteria growing in biofilms are more resistant to antibiotics and disinfectants than planktonic cells, and the resistance increases with the age of the biofilm. Bacterial biofilms also exhibit increased physical resistance towards desiccation, extreme temperatures or light. As mentioned, biofilm formation causes industrial, environmental and medical problems, and the difficulties in cleaning and disinfection of bacterial biofilm with chemicals is a major concern in many industries. Furthermore, the trend towards milder disinfection and cleaning compositions may increase the current insufficient cleaning of surfaces covered with biofilm.

The prior art contains examples of instantly generating iodine in order to provide a germicidal activity. U.S. Pat. No. 3,232,869 teaches a method for purifying and disinfecting aqueous liquids with free molecular iodine, where the iodine is provided by quantitatively oxidizing iodide ion into free molecular iodine with persulfates in the pH range between 7 and 8. This patent requires the use of a stoichiometric amount of either iodide or persulfate to yield a free iodine concentration of 0.1 to 1.0 ppm of free molecular iodine.

U.S. Pat. No. 3,215,627 discloses a method for use in the disinfection of swimming pools. A pH range of 7 to 8 is taught as critical to both U.S. Pat. Nos. 3,232,869 and 3,215,627. The range of free molecular iodine that is generated according to the method of the '627 patent is between 0.2 and 0.4 ppm. This patent also teaches that an iodide bank is of no value because iodine release is erratic and unpredictable and because it is not possible to achieve or maintain a desired iodine level.

U.S. Pat. No. 3,215,627 and U.S. Pat. No. 3,232,869 identify a concentration range of 0.1 to 1.0 ppm of iodide ion as the practical concentration range. This concentration of iodide equates to a theoretical maximum free molecular iodide concentration of 0.85 ppm. Moreover, both the '627 and '869 patents teach that a pH in the range of 7 to 8 is critical.

### SUMMARY OF THE INVENTION

The present invention provides for a method for disinfecting, removing biofilm and inactivating endotoxins in connection with medical devices, biological environments and in households. More particularly, there is provided compositions which are non-toxic and provide a source of available iodine and an oxidizing agent to result in a broad spectrum of germicidal activity and to inactivate endotoxins.

The method of the invention relates to applying to a surface to be treated a composition with a pH of 2.3-6 that is free of a persulfate and comprises:
A. an effective amount of a monobasic iodide salt to provide at least 30 ppm of available iodine to said composition, preferably about 80 to 500 ppm;
B. an organic acid having up to eight atoms;
C. an oxidizing agent'
D. a buffer, and
E. an aqueous solvent.

Optionally, an inorganic magnesium salt can be added to enhance the germicidal activity or can be present as the iodide.

The compositions can be used in the form of a spray, dip, brushed or other means common in the art.

It is a general object of the invention to provide a method and a composition for disinfecting surfaces, inactivating endotoxin and remove biofilm in households, on skin and on medical equipment.

It is another object of the invention to provide an effective and environmentally safe method for eliminating biofilm, the living pathogens and the endotoxins on an organic or inorganic surface.

It is still another object of the invention to disinfect medical equipment such as hemodialysis equipment and waterlines.

It is yet another object of the invention to provide a composition which is bactericidal, pseudomonacidal, virucidal, tuberculecidal, fungicidal and inactivates endotoxins.

### DESCRIPTION OF THE PREFERRED EMBODIMENTS

According to the present invention, there is provided a method and a composition for removing biofilm and inactivating endotoxins therein that generates a safe level of microbicidal molecular iodine and does not contain persulfates. The compositions of the invention consist essentially of:
A. an effective amount of a monobasic iodide salt to provide at least 30 ppm of available iodine, preferably about 80 to 500 ppm;
B. an organic acid having up to eight carbon atoms;
C. an oxidizing agent;
D. a buffer,
E. an aqueous solvent, and optionally,
F. an inorganic magnesium salt.
   A preferred composition of the invention consists of:
1) at least about 1 to 5% by weight of a monobasic iodide salt which is an inorganic metal salt, preferably an alkali metal salt;
2) about 10 to 20% by weight of an organic acid having up to eight carbon atoms, more particularly selected from the group consisting of citric acid, ascorbic acid, oxalic acid, and the alkali salts thereof;
3) about 0.5 to 5% by weight of an oxidizing agent selected from the group consisting of alkali salts of peroxide, sodium percarbonate, sodium perborate, urea hydrogen peroxide, peroxidase, ascorbic acid, and citric acid;
4) about 5 to 15% by weight of a buffer, which is preferably a phosphate buffer and the remainder
5) an aqueous solvent.

The composition is buffered to a pH of 2.2 to 6.0, preferably about 3.0 to 3.6. There is an available iodine of at least 30 pm, preferably about 80 to 300 ppm.

The solvent can comprise at least 50% water with an alkanol having 1 to 4 carbon atoms or can be 100% water, preferably the alcohol is ethanol and/or isopropanol.

Optional ingredients can be a magnesium salt, namely magnesium iodide or magnesium sulfate. The magnesium iodide can be used to provide the source of molecular iodine alone or in combination with sodium or potassium iodide. The magnesium salts are also anti-microbial.

In general, the required pH to the overall composition can be any organic or inorganic acid which does not chemically react with the other components, such as hydrochloric acid, phosphate salts, phosphoric acid, sulfuric acid, citric acid, acetic acid, preferably the organic acids and/or phosphate salts are utilized.

The lower pH has the greatest amount of iodine in parts per million.

Buffering agents may be utilized to maintain pH within the desired range of 2.3 to 6.0, or within the more preferred range of 3.0 to 3.5. Suitable buffering agents for inclusion in the compositions of the invention include potassium phosphate, mono or dibasic, glycine-glycine-HCl, potassium hydrogen phthalate-phthalic acid, citric acid-Na₂HPO₄, citric acid-KH₂PO₄—H₃BO₃-diethylbarbituric acid-NaOH, citric acid-sodium citrate, dimethylglutaric acid-sodium dimethylglutarate, acetic acid-sodium acetate, succinic acid-sodium succinate, potassium hydrogen phthalate-dipotassium phthalate, sodium cacodylate-cacodylic acid, sodium hydrogen maleate-disodium maleate, Na₂HPO₄—NaH₂PO₄, sodium bicarbonate-5% CO₂, imidazole-imidazole HCl, boric acid-sodium borate, and the following buffers known to one skilled in the art: Tris, MES, BIS-TRIS, ADA, ACES and PIPES. Enough buffer is added to maintain the pH below 6.0 or, if preferred, within a defined pH limit that is less than pH 6.0. In general, a buffer concentration of at least 5 millimolar is utilized.

Aqueous mediums suitable for use in the present invention include water, mixtures of water and alcohols (such as ethanol, and isopropanol), or mixtures of water and other water-miscible solvents. In general, an aqueous medium will be capable of dissolving iodide salts and will not react rapidly with free molecular iodine. In preferred embodiments, the aqueous medium is substantially non-toxic. In preferred embodiments, the aqueous medium is at least 50% water by volume so as to be synergistic with the alcohol as a disinfecting agent.

The compositions of the present invention can be used without any further additive to disinfect medical apparatuses and devises by spraying or dipping, or otherwise treating the medical equipment to kill bacteria, remove biofilm and inactivate endotoxin.

In households, the composition can be placed into an aerosol spray to sanitize bathrooms, kitchen surfaces and sickrooms.

The composition can also be formulated into a germicidal detergent composition utilizing an amphoteric surfactant. However, additional non-amphoteric sulfactants can be utilized to increase detergency. Any of the conventional non-amphoteric surfactants such as condensates of alkylene oxide, e.g. ethylene or propylene oxide and a hydrophobic compound can be used. Among commercially available surfactants is included DERPHAT 170C™ from General Mills, Inc., Illinois, U.S.

For personal use, a small aerosol container which delivers a fine spray is a practical packaging for the composition. For commercial packaging a hydrocarbon propellant is preferred to meet environmental quality standards. Another preparation can be the saturation of a woven or non-woven wipe which is sealed for one time usage in an air tight package which is suitable to wipe telephones, hands and hard surfaces.

The composition can be formed in a gel to treat body parts or in the form of a wipe to disinfect hands or other body parts.

The following examples illustrate the invention:

### EXAMPLE 1

A disinfectant solution having a pH of 2.2-3.6 is prepared by admixing the following ingredients:

| Ingredient | Wt. % |
|---|---|
| Sodium iodide | 2.45 |
| Potassium phosphate | |
| Monohydrate | 10.00 |
| Citric acid, anhydrous | 15.80 |
| Sodium perborate | |
| Monohydrate | 0.42 |
| Deionized water | q.s. |
| | 100% |

2% of TRITONX-100 (octyl phenoxy polyethoxyethanol) of Sigma Chemical Co. may be added to provide detergency.

The formula produces a concentrate with about 10,000 ppm of iodine.

### EXAMPLE 2

The efficacy dilutions of the formula of Example 1 in removing biofilm from silicone tubing in a model system that simulated hemodialysis waterlines was determined following the procedure of Marion-Ferey et al, J. Hospital Infection, 53, 64-71, 2003.

Biofilm was formed in the tubing for a period of five days, after which samples were collected for the determination of hetrotrophic plate count (HPC) and endotoxin levels.

| Experiment | Reactor HPC (CFU/ml) | Reactor Endotoxin (EU/ml) |
|---|---|---|
| 1 | 4.72 × 10⁶ | 431 |
| 2 | 2.50 × 10⁸ | 513 |
| 3 | 1.33 × 10⁷ | 274 |
| 4 | 1.56 × 10⁷ | 480 |

Tubing in Experiments 1-3 were treated with the formula of Example 1 for 8 hours under static conditions. The results were as follows:

| Experiment | HPC Log Reduction | Endotoxin % Reduction |
|---|---|---|
| 1 | >3.0 | 48 |
| 2 | >5.0 | 41 |
| 3 | 4.9 | 42 |

The tubing in Experiment 4 with treatment at times of 2, 4 and 8 hours. The results were as follows:

| Treatment Time (hours) | HPC Log Reduction | Endotoxin % Reduction |
|---|---|---|
| 2 | 6.2 | 68 |
| 4 | 6.1 | 32 |
| 8 | 5.8 | 94 |

Two hours of treatment was sufficient to achieve a maximum reduction in HPC. The percentage reduction in endotoxin levels indicate that the 8 hour treatment was most effective. Endotoxins in the control tubing samples ranged from 195 to 404 EU/cm².

### EXAMPLE 3

### Object: To study the effect of iodine formulation Example 1 at three different pH levels on metabolic activity of cells in Candida albicans biofilm.

Methods: The three iodine formulations used in this experiment were: 1. Iodine pH 5.0, 80 ppm Iodine; 2. Iodine pH 6.4, 80 ppm Iodine; and 3. Iodine pH 3.30, 150 ppm Iodine.

Candida albicans biofilm was grown in three 96 wells micro-titer plates for 24 hours. The wells were carefully emptied and washed three times with phosphate-buffered saline to remove unattached cells. In each plate, one row of eight wells was used as control. In other six rows of eight wells, the biofilm was exposed to 15 or 30 uL of each of the three formulations listed above for one, five and twenty minutes. After the exposure time, fluids from the wells were carefully aspirated and the biofilms were washed repeatedly with 100, 50 and 50 uL of PBS. A semi-quantitative measure of biofilm formation was determined by using the XTT reduction assay of Ramage, G. et al (2001) Standardized method for in vitro antifungal susceptibility testing of Candida albicans biofilm, antimicrobial agents and chemotherapy. 9:2475-2479. The percent inhibition of Candida albicans biofilm by these iodine formulations was compared to the biofilm in the control wells.

### Results: The following table represents the summary of the percent inhibition of C. albicans biofilm formation by the three formulations at one-half or full strength and for the three exposure times.

| | Percent Inhibition | | |
|---|---|---|---|
| Exposure Time: | 1 minute | 5 minutes | 20 minutes |
| Cells + Iodine pH 5.0 - 30 uL | 48.00 | 63.89 | 66.96 |
| Cells + Iodine pH 6.4 - 30 uL | 78.57 | 93.33 | 95.21 |
| Cells + Iodine pH 3.3 - 30 uL | 84.44 | 95.74 | 94.84 |
| Cells + Iodine pH 5.0 - 15 uL | 38.89 | 29.47 | 50.28 |
| Cells + Iodine pH 6.4 - 15 uL | 50.44 | 54.77 | 64.77 |
| Cells + Iodine pH 3.3 - 15 uL | 58.22 | 53.12 | 50.42 |

The percent inhibition by the Iodine formulation pH 3.3 at full strength was 84.44, 95.74 and 94.84 after 1, 5, and 20 minute exposure times, respectively, and that by the full strength iodine formulation at pH 6.4 was 78.57, 93.33 and 95.21 at the same exposure times respectively. The percent inhibition was similar at 5 and 20 minute exposures by both of these formulations. The percent inhibition by these two iodine formulations at one-half strength was between 29 to 65% at all three exposure times. The percent inhibition of C.albicans biofilm formation by the iodine formulation at pH 5.0 was 48.00, 63.89 and 66.96 after 1, 5, and 20 minute exposure at full strength and 38.89, 29.47 and 50.28 at one-half its strength.

Conclusions: The iodine formulation pH 3.3 was found to be more effective in inhibiting Candida albicans biofilm formation at a 5 minute exposure time. The effect of iodine formulations pH 5.0 was less in inhibiting Candida albicans biofilm formation.

What is claimed is:

1. A method for reducing biofilm, disinfecting and inactivating endotoxin on a surface and in solution which comprises treating said surface and solution with a composition which is free of persulfate ions and consists essentially of:
a) an effective amount of a monobasic salt to provide at least 30 ppm of available iodine.
b) about 10 to 20% by weight of an organic acid having up to eight carbon atoms;
c) about 0.5 to 5% by weight of an oxidizing agent;
d) about 5 to 15% by weight of a buffer; and the remainder
e) an aqueous solvent,
said composition having a pH of 2.3 to 6.0.

2. The method of claim 1 wherein said surface comprises medical equipment.

3. The method of claim 2 wherein said surface comprises parts of a hemodialysis system.

4. The method of claim 1 wherein said composition is sprayed.

5. The method of claim 1 wherein said composition includes an amphoteric surfactant.

6. The method of claim 1 wherein said composition includes a salt selected from the group consisting of magnesium iodide, magnesium chloride, and magnesium sulfate.

7. The method of claim 1 wherein said monobasic salt is selected from the group consisting of sodium iodide, potassium iodide, magnesium iodide and calcium iodide.

8. The method of claim 1 wherein said solvent is water.

9. The method of claim 1 wherein the pH of the composition is about 3.0 to 3.5.

10. The method of claim 1 wherein endotoxins are inactivated on medical equipment.

11. The method of claim 1 wherein said surface is a skin surface.

12. The method of claim 1 wherein said composition comprises a gel.

13. The method of claim 1 wherein said buffer comprises a phosphate.

14. The method of claim 1 wherein said oxidizing agent comprises sodium perborate.

15. The method of claim 1 wherein said composition comprises:
a) about 1 to 5% by weight of a monobasic iodide salt;
b) about 10 to 20% by weight of an organic acid having up to eight carbon atoms;
c) about 0.5 to 5% of an oxidizing agent
d) about 5 to 15% by weight of a phosphate. buffer, and the remainder
e) a lower alkanol solvent containing at least 50% water.
